(19) United States
(12) Patent Application Publication
(10) Pub. No.: US 2006/0252983 A1
(43) Pub. Date: Nov. 9, 2006

(54) Dynamically adjustable gastric implants and methods of treating obesity using dynamically adjustable gastric implants

(76) Inventors: Nicholas J. Lembo, Atlanta, GA (US); Shawn Moaddeb, Irvine, CA (US); Emanuel Shaoulian, Newport Beach, CA (US); Samuel M. Shaolian, Newport Beach, CA (US); Michael R. Henson, Coto de Caza, CA (US)

Correspondence Address: KNOBBE MARTENS OLSON & BEAR LLP, 2040 MAIN STREET, FOURTEENTH FLOOR, IRVINE, CA, 92614, US

(21) Appl. No.: 11/351,788

(22) Filed: Feb. 10, 2006

Related U.S. Application Data
(60) Provisional application No. 60/652,133, filed on Feb. 11, 2005.
(60) Provisional application No. 60/652,466, filed on Feb. 11, 2005.
(60) Provisional application No. 60/701,805, filed on Jul. 22, 2005.

(51) Int. Cl. A61F 2/00; A61B 17/08; A61F 13/00

(52) U.S. Cl. 600/37; 606/151

(57) ABSTRACT

Implants for treatment of obesity may be placed within the stomach and/or esophagus or around the outside surface of the stomach and/or esophagus. The implants comprise at least a portion constructed of a shape memory material. The implants are thus adapted to be implanted in a deformed shape, and then to be transformed through the application of activation energy into a memorized shape. The shape and/or size transformation induces a change in shape of the stomach and/or esophagus, thus altering the normal digestive path of food entering the patient's gastrointestinal tract.

### CROSS-REFERENCE TO RELATED APPLICATIONS

This application claims priority to provisional application Ser. No. 60/652,133, filed on Feb. 11, 2005, provisional application Ser. No. 60/652,466, filed on Feb. 11, 2005, and provisional application Ser. No. 60/701,805, filed on Jul. 22, 2005. The entire contents of each of the priority applications are hereby incorporated by reference.

### BACKGROUND OF THE INVENTION

1. Field of the Invention

The present invention relates to a system and method for dynamically reshaping and resizing the stomach and/or esophagus using an implant or implants within or around the outside of the stomach and/or esophagus and externally or internally activating the implant(s) to induce a change in shape and/or size of the implant(s).

2. Description of the Related Art

Bariatrics is the branch of medicine concerned with the management of obesity and allied diseases. There are two main categories of bariatric surgery techniques available today. Restrictive techniques reduce the amount of food that can be consumed by restricting the size and/or capacity of the stomach. Malabsorptive techniques alter and/or shorten the digestive tract to decrease the absorption of calories and nutrients. Some surgeries are just restrictive, while others are both restrictive and malabsorptive.

One common obesity surgery is the Roux-en-Y Gastric Bypass (often known only as a “Gastric Bypass”). During this type of operation, the surgeon permanently changes the shape of the stomach by surgically reducing (cutting or stapling) its size to create an egg-sized gastric pouch or “new stomach”. The rest of the stomach is then divided and separated from this new stomach pouch, greatly reducing the amount of food that can be consumed after surgery. In addition to reducing the actual size of the stomach, a significant portion of the digestive tract is bypassed and the new stomach pouch is reconnected directly to the bypassed segment of small intestine. This operation, therefore, is both a restrictive and malabsorptive procedure, because it limits the amount of food that one can eat and the amount of calories and nutrition that are absorbed or digested by the body. Once completed, gastric bypass surgery is essentially irreversible. Some of the major risks associated with the Roux-en-Y Gastric Bypass procedure include: bleeding, infection, pulmonary embolus, anastomotic stricture or leak, anemia, ulcer, hernia, gastric distention, bowel obstruction and death.

Another common obesity surgery is known as Vertical Banded Gastroplasty (“VBG”), or “stomach stapling.” In a gastroplasty procedure, the surgeon staples the upper stomach to create a small, thumb-sized stomach pouch, reducing the quantity of food that the stomach can hold to about 1-2 ounces. The outlet of this pouch is then restricted by a band that significantly slows the emptying of the pouch to the lower part of the stomach. Aside from the creation of a small stomach pouch, there is no other significant change made to the gastrointestinal tract. So while the amount of food the stomach can contain is reduced, the stomach continues to digest nutrients and calories in a normal way. This procedure is purely restrictive; there is no malabsorptive effect. Following this operation, many patients have reported feeling full but not satisfied after eating a small amount of food. As a result, some patients have attempted to get around this effect by eating more or by eating gradually all day long. These practices can result in vomiting, tearing of the staple line, or simply reduced weight loss. Major risks associated with VBG include: unsatisfactory weight loss or weight regain, vomiting, band erosion, band slippage, breakdown of staple line, anastomotic leak, and intestinal obstruction.

A third procedure, the Duodenal Switch, is less common. It is a modification of the Biliopancreatic Diversion or “Scopinaro Procedure.” While this procedure is considered by many to be the most powerful weight loss operation currently available, it is also accompanied by significant long-term nutritional deficiencies in some patients. Many surgeons have stopped performing this procedure due to the serious associated nutritional risks.

In the Duodenal Switch procedure, the surgeon removes about 80% of the stomach, leaving a very small new stomach pouch. The beginning portion of the small intestine is then removed, and the severed end portions of the small intestine are connected to one another near the end of the small intestine and the beginning of the large intestine or colon. Through this procedure a large portion of the intestinal tract is bypassed so that the digestive enzymes (bile and pancreatic juices) are diverted away from the food stream until very late in the passage through the intestine. The effect of this procedure is that only a small portion of the total calories that are consumed are actually digested or absorbed. This irreversible procedure, therefore, is both restrictive (the capacity of the stomach is greatly reduced) and malabsorptive (the digestive tract is shortened, severely limiting absorption of calories and nutrition). Because of the very significant malabsorptive component of this operation, patients must strictly adhere to dietary instructions including taking daily vitamin supplements, consuming sufficient protein and limiting fat intake. Some patients also experience frequent large bowel movements, which have a strong odor. The major risks associated with the Duodenal Switch are: bleeding, infection, pulmonary embolus, loss of too much weight, vitamin deficiency, protein malnutrition, anastomotic leak or stricture, bowel obstruction, hernia, nausea/vomiting, heartburn, food intolerances, kidney stone or gallstone formation, severe diarrhea and death.

One relatively new and less invasive form of bariatric surgery is Adjustable Gastric Banding. Through this procedure the surgeon places a band around an upper part of the stomach to divide the stomach into two parts, including a small pouch in the upper part of the stomach. The small upper stomach pouch can only hold a small amount of food. The remainder of the stomach lies below the band. The two parts are connected by means of a small opening called a stoma. Risks associated with Gastric Banding are significantly less than other forms of bariatric surgery, since this surgery does not involve opening of the gastric cavity. There is no cutting, stapling or bypassing.

The LAP-BAND® Adjustable Gastric Banding System (Inamed) is one current product used in the Adjustable Gastric Banding procedure. The LAP-BAND® system, illustrated in FIG. 1, comprises a silicon band 50, which is essentially an annular-shaped balloon. The surgeon places the silicon band around the upper part of the stomach 52, as described above. The LAP-BAND® system further comprises a port 54 that is placed under the skin, and tubing 56 that provides fluid communication between the port and the band. A physician can inflate the band by injecting a fluid (such as saline) into the band through the port. As the band inflates, the size of the stoma shrinks, thus further limiting the rate at which food can pass from the upper stomach pouch 58 to the lower part of the stomach. The physician can also deflate the band, and thereby increase the size of the stoma, by withdrawing the fluid from the band through the port. The physician inflates and deflates the band by piercing the port, through the skin, with a fine-gauge needle.

### SUMMARY OF THE INVENTION

The preferred embodiments of the present gastric implants and methods have several features, no single one of which is solely responsible for their desirable attributes. Without limiting the scope of these gastric implants and methods as expressed by the claims that follow, their more prominent features will now be discussed briefly. After considering this discussion, and particularly after reading the section entitled “Detailed Description of the Preferred Embodiments,” one will understand how the features of the preferred embodiments provide advantages, which include dynamic adjustability through minimally invasive or completely noninvasive procedures.

One embodiment of the present gastric implants and methods comprises an adjustable gastric implant configured to be implanted within a stomach and/or an esophagus or around outer surfaces of the stomach and/or the esophagus. The implant comprises an implant body having at least a portion thereof formed from a material having a shape memory. The implant body is configured to transform under the influence of an activation energy from a pre-activation configuration to a post-activation configuration. The implant reshapes the stomach and/or the esophagus in transforming from the pre-activation configuration to the post-activation configuration.

In another embodiment, the implant is shaped substantially as at least one of a ring, an oval, a C, a D, a U, an S, a helix, a coil, a tube, a tubular cage, and a wire stent.

In another embodiment, the implant further comprises apparatus configured to facilitate the securement of the implant to the stomach and/or the esophagus.

In another embodiment, the apparatus for facilitating the securement of the implant to the stomach and/or the esophagus comprises at least one of a suture hole, a suture ring, a hook, a barb, and an anchor.

In another embodiment, the implant body comprises a core having at least a portion thereof formed from a material having a shape memory, and a cover disposed over at least a portion of the core.

In another embodiment, the implant further comprises apparatus configured to resist a tendency of the implant to transform from the post-activation configuration to the pre-activation configuration.

In another embodiment, the apparatus for resisting transformation comprises a ratchet.

In another embodiment, at least the portion of the implant body that is formed from a material having a shape memory comprises at least one of a metal, a metal alloy, a nickel titanium alloy, and a shape memory polymer.

In another embodiment, at least the portion of the implant body that is formed from a material having a shape memory comprises at least one of Fe—C, Fe—Pd, Fe—Mn—Si, Co—Mn, Fe—Co—Ni—Ti, Ni—Mn—Ga, Ni₂MnGa, and Co—Ni—Al.

In another embodiment, the implant is configured to transform from the pre-activation configuration to the post-activation configuration in response to at least one of a magnetic resonance imaging energy, high-intensity focused ultrasound energy, radio frequency energy, x-ray energy, microwave energy, light energy, electric field energy, magnetic field energy, inductive heating, and conductive heating.

In another embodiment, the implant body comprises a frame portion and a band portion, the frame portion and the band portion each being formed of a material that does not have a shape memory.

In another embodiment, the implant body comprises a frame portion and a band portion, the frame portion and the band portion each being formed of a material that does not have a shape memory, and further comprises a shape memory portion located intermediate the frame portion and the band portion.

Another embodiment of the present gastric implants and methods comprises a method for treating obesity. The method comprises the steps of placing an adjustable gastric implant comprising a shape memory material within or around an outside surface of a patient's stomach and/or esophagus, and applying an activation energy to the shape memory material. The activation energy induces a transformation in shape and/or size of the implant, thereby reshaping the stomach and/or esophagus.

In another embodiment of the method, the implant is shaped substantially as at least one of a ring, an oval, a C, a D, a U, an S, a helix, a coil, a tube, a tubular cage, and a wire stent.

In another embodiment of the method, the implant comprises at least one of a metal, a metal alloy, a nickel titanium alloy, and a shape memory polymer.

In another embodiment of the method, the implant comprises at least one of Fe—C, Fe—Pd, Fe—Mn—Si, Co—Mn, Fe—Co—Ni—Ti, Ni—Mn—Ga, Ni₂MnGa, and Co—Ni—Al.

In another embodiment of the method, the activation energy comprises at least one of magnetic resonance imaging energy, high-intensity focused ultrasound energy, radio frequency energy, x-ray energy, microwave energy, light energy, electric field energy, magnetic field energy, inductive heating, and conductive heating.

Another embodiment of the present gastric implants and methods comprises an adjustable gastric implant. The implant comprises means for engaging at least one of a stomach and/or an esophagus. Said means comprises a shape memory material. Said means is configured to change shape from a first configuration to a second configuration in response to an activation energy. Said means is configured to reshape said stomach and/or said esophagus in changing shape from said first configuration to said second configuration.

### BRIEF DESCRIPTION OF THE DRAWINGS

The preferred embodiments of the present gastric implants and methods, illustrating their features, will now be discussed in detail. These embodiments depict the novel and non-obvious gastric implants shown in the accompanying drawings, which are for illustrative purposes only. These drawings include the following figures, in which like numerals indicate like parts:

FIG. 1 is a front elevational view of a stomach that has undergone a Gastric Banding procedure using the prior art LAP-BAND® Adjustable Gastric Banding System;

FIG. 2 is a front elevational view of a stomach that has undergone a Gastric Banding procedure using one embodiment of the present dynamically adjustable gastric implants;

FIG. 3 is a front elevational view of the stomach of FIG. 2 after the implant has been adjusted;

FIG. 4 is a front elevational view of a stomach that has undergone a Gastric Banding procedure using another embodiment of the present dynamically adjustable gastric implants;

FIG. 5 is a front perspective view of one embodiment of the present dynamically adjustable gastric implants;

FIG. 6 is a front perspective view of the implant of FIG. 5 after the implant has been adjusted;

FIG. 7 is a front perspective view of the implant of FIG. 5 after the implant has been further adjusted from the configuration of FIG. 6;

FIG. 8 is a top plan view of another embodiment of the present dynamically adjustable gastric implants, illustrating the implant in a pre-adjusted configuration;

FIG. 9 is a top plan view of the implant of FIG. 8, illustrating the implant in a post-adjusted configuration;

FIG. 10 is a top plan view of another embodiment of the present dynamically adjustable gastric implants;

FIG. 11 is a top plan view of another embodiment of the present dynamically adjustable gastric implants;

FIG. 12 is a top plan view of another embodiment of the present dynamically adjustable gastric implants;

FIG. 13 is a top plan view of another embodiment of the present dynamically adjustable gastric implants;

FIG. 14 is a top plan view of another embodiment of the present dynamically adjustable gastric implants;

FIG. 15 is a detail view of the portion of the implant of FIG. 14 indicated by the line 15-15;

FIG. 16 is a top plan view of another embodiment of the present dynamically adjustable gastric implants, illustrating the implant in a pre-adjusted configuration;

FIG. 17 is a top plan view of the implant of FIG. 16, illustrating the implant in a post-adjusted configuration;

FIG. 18 is a top plan view of the implant of FIGS. 16 and 17, illustrating the pre-adjusted and post-adjusted configurations superimposed upon one another;

FIG. 19 is a top plan view of another embodiment of the present dynamically adjustable gastric implants, illustrating the implant in a pre-adjusted configuration;

FIG. 20 is a top plan view of the implant of FIG. 19, illustrating the implant in a post-adjusted configuration;

FIG. 21 is a front elevational view of another embodiment of the present dynamically adjustable gastric implants and a stomach, illustrating a configuration of the implant and stomach after activation of the implant;

FIG. 22 is a front elevational view of another embodiment of the present dynamically adjustable gastric implants and a stomach, illustrating a configuration of the implant and stomach after activation of the implant;

FIG. 23 is a front elevational view of another embodiment of the present dynamically adjustable gastric implants and a stomach, illustrating a configuration of the implant and stomach after activation of the implant;

FIG. 24 is a top plan view of another embodiment of the present dynamically adjustable gastric implants, illustrating the implant in a pre-adjusted configuration;

FIG. 25 is a top plan view of the implant of FIG. 24, illustrating the implant in a post-adjusted configuration;

FIG. 26 is a front perspective view of another embodiment of the present dynamically adjustable gastric implants;

FIG. 27 is a front elevational view of another embodiment of the present dynamically adjustable gastric implants;

FIG. 28 is a front elevational view of another embodiment of the present dynamically adjustable gastric implants, illustrating several different sizes of the embodiment;

FIG. 29 is a front perspective view of another embodiment of the present dynamically adjustable gastric implants;

FIG. 30 is a front elevational view of a stomach and esophagus, illustrating schematically one possible configuration for implantation of any of the implants of FIGS. 26-29;

FIG. 31 is a detail view of a portion of another embodiment of the present dynamically adjustable gastric implants;

FIG. 32 is a detail view of the portion of FIG. 31 after the implant has been adjusted;

FIG. 33 is a front elevational view of a patient and another embodiment of the present dynamically adjustable gastric implants, illustrating one method of adjusting the implant using direct application of electrical impulses; and

FIG. 34 is a front elevational view of one step in a method of implanting any of the present implants using a balloon catheter.

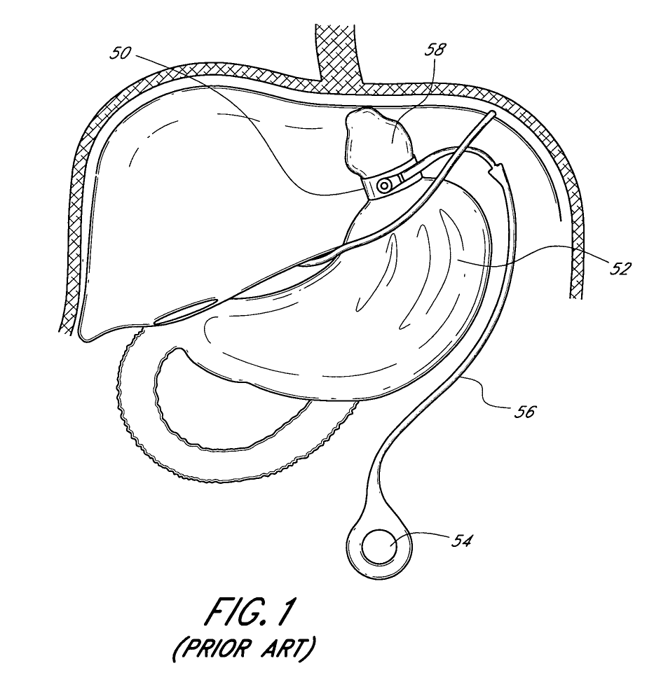

### DETAILED DESCRIPTION OF THE PREFERRED EMBODIMENTS

The present embodiments include gastric implants and methods for dynamically adjusting the stomach and/or esophagus of a patient to treat obesity. As used herein, the term “gastric” refers not only to the stomach, but also to the esophagus. Accordingly, “gastric implant” describes not only an implant that is configured for implantation within or around the outside of the stomach, but also an implant that is configured for implantation within or around the outside of the esophagus.

In certain embodiments, an adjustable implant is implanted into the body of a patient such as a human or other animal. The adjustable implant may be disposed around the stomach, or within the stomach. The adjustable implant may also be disposed around the esophagus, or within the esophagus. The implant may be selected from one or more shapes comprising a ring shape (note that as used herein the term “ring” comprises both circular and non-circular shapes, and both open and closed configurations), an oval shape, a C-shape, a D-shape, a U-shape, an S-shape, a helical or coil shape, a cage shape, a wire stent shape and other shapes. The implant may be implanted through an incision during a traditional open procedure, or endoscopically, or laparoscopically, or percutaneously, or through another type of procedure, as those of skill in the art will appreciate.

A variety of different implant locations are described below, including entirely within or around the stomach, and at the junction of the esophagus and the stomach. Those of skill in the art will appreciate that the present implants may be implanted anywhere within or around the stomach and/or the esophagus, and that multiple implants can be placed at different locations within the stomach and/or the esophagus. Further, the implants described herein can also be used in combination with other surgical procedures, such as Gastric Bypass, VBG, Duodenal Switch, etc.

The size and/or configuration of the present implants can be adjusted post-implantation through one of many techniques, including minimally invasive techniques and completely non-invasive techniques. For example, minimally invasive techniques include endoscopic, laparoscopic, percutaneous, etc. Completely non-invasive techniques include magnetic resonance imaging (MRI), application of high-intensity focused ultrasound (HIFU), inductive heating, a combination of these methods, etc. The implant may be adjusted at a time shortly after implantation in order to constrict and/or expand a portion of the stomach. The implant may also be adjusted at a later time in order to further constrict and/or expand the stomach and/or to allow a previously constricted portion of the stomach to expand and/or to allow a previously expanded portion of the stomach to constrict. As used herein, “post-implantation” refers to a time after implanting the implant and closing the body opening through which the implant was introduced into the patient's body.

In certain embodiments, the implant comprises a shape memory material that is responsive to changes in temperature and/or exposure to a magnetic field. Shape memory is the ability of a material to regain its shape after deformation. Shape memory materials include polymers, metals, metal alloys and ferromagnetic alloys. The implant may be adjusted in vivo by applying an energy source to activate the shape memory material and cause it to change to a memorized shape. The energy source may include, for example, radio frequency (RF) energy, x-ray energy, microwave energy, ultrasonic energy such as focused ultrasound, HIFU energy, light energy, electric field energy, magnetic field energy, combinations of the foregoing, or the like. For example, one embodiment of electromagnetic radiation that is useful is infrared energy having a wavelength in a range between approximately 750 nanometers and approximately 1600 nanometers. This type of infrared radiation may be produced efficiently by a solid state diode laser. In certain embodiments, the implant may be selectively heated using short pulses of energy having an on and off period between each cycle. The energy pulses provide segmental heating, which allows segmental adjustment of portions of the implant without adjusting the entire implant.

In certain embodiments, the implant may include an energy absorbing material to increase heating efficiency and localize heating in the area of the shape memory material. Thus, damage to the surrounding tissue can be reduced or eliminated. Energy absorbing materials for light or laser activation energy may include nanoshells, nanospheres and the like, particularly where infrared laser energy is used to energize the material. Such nanoparticles may be made from a dielectric, such as silica, coated with an ultra thin layer of a conductor, such as gold, and be selectively tuned to absorb a particular frequency of electromagnetic radiation. In certain such embodiments, the nanoparticles range in size between about 5 nanometers and about 20 nanometers and can be suspended in a suitable material or solution, such as saline solution. Coatings comprising nanotubes or nanoparticles can also be used to absorb energy from, for example, HIFU, MRI, inductive heating, or the like. In the case of MRI, the coating might include a specific resonance frequency other than the 64 MHz that is typically used in MRI. Thus, the implant can be imaged and controllably adjusted in size and/or shape by using two or more different frequencies of energy simultaneously. A tuneable frequency can be used to better direct activation energy without impacting the image quality.

In other embodiments, thin film deposition or other coating techniques such as sputtering, reactive sputtering, metal ion implantation, physical vapor deposition, and chemical deposition can be used to cover portions or all of the implant. Such coatings can be either solid or microporous. When HIFU energy is used, for example, a microporous structure may trap and direct the HIFU energy toward the shape memory material. The coating improves thermal conduction and heat removal. In certain embodiments, the coating also enhances radio-opacity of the implant. Coating materials can be selected from various groups of biocompatible organic or non-organic, metallic or non-metallic materials such as titanium nitride (TiN), iridium oxide (Irox), carbon, graphite, ceramic, platinum black, titanium carbide (TiC) and other materials used for pacemaker electrodes or implantable pacemaker leads. Other materials discussed herein or known in the art can also be used to absorb energy.

In addition, or in other embodiments, fine conductive wires such as platinum coated copper, titanium, tantalum, stainless steel, gold, or the like, may be wrapped around the shape memory material to allow focused and rapid heating of the shape memory material while reducing undesired heating of surrounding tissues.

In certain embodiments, the energy source is applied surgically either during implantation or at a later time. For example, the shape memory material can be heated during implantation of the implant by touching the implant with a warm object. As another example, the energy source can be surgically applied after the implant has been implanted by inserting a catheter into the patient's body and applying the energy through the catheter. The catheter may be inserted percutaneously, or through a peroral transgastric procedure, for example. Various types of energy, such as ultrasound, microwave energy, RF energy, light energy or thermal energy (e.g., from a heating element using resistance heating), can be transferred to the shape memory material through a catheter positioned on or near the shape memory material. Alternatively, thermal energy can be provided to the shape memory material by injecting a heated fluid through a catheter or circulating the heated fluid in a balloon through the catheter placed in close proximity to the shape memory material. As another example, the shape memory material can be coated with a photodynamic absorbing material that is activated to heat the shape memory material when illuminated by light from a laser diode or directed to the coating through fiber optic elements in a catheter. In certain such embodiments, the photodynamic absorbing material includes one or more drugs that are released when illuminated by the laser light.

In certain embodiments, a removable subcutaneous electrode or coil couples energy from a dedicated activation unit. In certain such embodiments, the removable subcutaneous electrode provides telemetry and power transmission between the system and the implant. The subcutaneous removable electrode allows more efficient coupling of energy to the implant with minimum or reduced power loss. In certain embodiments, the subcutaneous energy is delivered via inductive coupling.

In other embodiments, the energy source is applied in a non-invasive manner from outside the patient's body. In certain such embodiments, the external energy source may be focused to provide directional heating to the shape memory material so as to reduce or minimize damage to the surrounding tissue. For example, in certain embodiments, a handheld or portable device comprising an electrically conductive coil generates an electromagnetic field that non-invasively penetrates the patient's body and induces a current in the implant. The current heats the implant and causes the shape memory material to transform to a memorized shape. In certain such embodiments, the implant may also comprise an electrically conductive coil wrapped around or embedded in the shape memory material. The externally generated electromagnetic field induces a current in the implant's coil, causing it to heat and transfer thermal energy to the shape memory material.

In certain other embodiments, an external HIFU transducer focuses ultrasound energy onto the implant to heat the shape memory material. In certain such embodiments, the external HIFU transducer is a handheld or portable device. The terms “HIFU,” “high intensity focused ultrasound” or “focused ultrasound” as used herein are broad terms and are used at least in their ordinary sense and include, without limitation, acoustic energy within a wide range of intensities and/or frequencies. For example, HIFU includes acoustic energy focused in a region, or focal zone, having an intensity and/or frequency that is considerably less than what is currently used for ablation in medical procedures. Thus, in certain such embodiments, the focused ultrasound is not destructive to the patient's organ tissue. In certain embodiments, HIFU includes acoustic energy within a frequency range of approximately 0.5 MHz and approximately 30 MHz and a power density within a range of approximately 1 W/cm² and approximately 500 W/cm².

In certain embodiments, the implant comprises an ultrasound absorbing material or hydro-gel material that allows focused and rapid heating when exposed to the ultrasound energy and transfers thermal energy to the shape memory material. In certain embodiments, a HIFU probe is used with an adaptive lens to compensate for movement within the body due to, for example, respiration. The adaptive lens has multiple focal point adjustments. In certain embodiments, a HIFU probe with adaptive capabilities comprises a phased array or linear configuration. In certain embodiments, an external HIFU probe comprises a lens configured to be placed between a patient's ribs to improve acoustic window penetration and reduce or minimize issues and challenges regarding passing through bones.

In certain embodiments, HIFU or other activation energy can be synchronized with an imaging device, such as MRI, ultrasound or X-ray, to allow visualization of the implant during HIFU activation. The imaging device may include an algorithm to display the area of interest for energy delivery. In addition, or in other embodiments, ultrasound imaging can be used to non-invasively monitor the temperature of tissue surrounding the implant by using principles of speed of sound shift and changes to tissue thermal expansion.

In certain embodiments, non-invasive energy is applied to the implant post-implantation using a Magnetic Resonance Imaging (MRI) device. In certain such embodiments, the shape memory material is activated by a constant magnetic field generated by the MRI device. In addition, or in other embodiments, the MRI device generates RF pulses that induce current in the implant and heat the shape memory material. The implant can include one or more coils and/or MRI energy absorbing material to increase the efficiency and directionality of the heating. Suitable energy absorbing materials for magnetic activation energy include particulates of ferromagnetic material. Suitable energy absorbing materials for RF energy include ferrite materials as well as other materials configured to absorb RF energy at resonant frequencies thereof.

In certain embodiments, the MRI device is used to determine the size of the implanted implant before, during and/or after the shape memory material is activated. In certain such embodiments, the MRI device generates RF pulses at a first frequency to heat the shape memory material and at a second frequency to image the implant. Thus, the size of the implant can be measured without heating the implant. In certain such embodiments, an MRI energy absorbing material heats sufficiently to activate the shape memory material when exposed to the first frequency and does not substantially heat when exposed to the second frequency. Other imaging techniques known in the art can also be used to determine the size of the implant including, for example, ultrasound imaging, computed tomography (CT) scanning, X-ray imaging, or the like. In certain embodiments, such imaging techniques also provide sufficient energy to activate the shape memory material.

As discussed above, shape memory materials include, for example, polymers, metals, and metal alloys including ferromagnetic alloys. Examples of shape memory polymers that are usable for certain embodiments of the present implant are disclosed by Langer, et al. in U.S. Pat. No. 6,720,402, issued Apr. 13, 2004, U.S. Pat. No. 6,388,043, issued May 14, 2002, and U.S. Pat. No. 6,160,084, issued Dec. 12, 2000, each of which are hereby incorporated by reference herein. Shape memory polymers respond to changes in temperature by changing to one or more permanent or memorized shapes. In certain embodiments, the shape memory polymer may be heated to a temperature between approximately 38 degrees Celsius and approximately 60 degrees Celsius. In certain other embodiments, the shape memory polymer may be heated to a temperature in a range between approximately 40 degrees Celsius and approximately 55 degrees Celsius. In certain embodiments, the shape memory polymer has a two-way shape memory effect wherein the shape memory polymer can be heated to change it to a first memorized shape and cooled to change it to a second memorized shape. The shape memory polymer can be cooled, for example, by inserting or circulating a cooled fluid through a catheter.

Shape memory polymers implanted in a patient's body can be heated non-invasively using, for example, external light energy sources such as infrared, near-infrared, ultraviolet, microwave and/or visible light sources. Preferably, the light energy is selected to increase absorption by the shape memory polymer and reduce absorption by the surrounding tissue. Thus, damage to the tissue surrounding the shape memory polymer is reduced when the shape memory polymer is heated to change its shape. In other embodiments, the shape memory polymer comprises gas bubbles or bubble containing liquids such as fluorocarbons and is heated by inducing a cavitation effect in the gas/liquid when exposed to HIFU energy. In other embodiments, the shape memory polymer may be heated using electromagnetic fields and may be coated with a material that absorbs electromagnetic fields.

Certain metal alloys have shape memory qualities and respond to changes in temperature and/or exposure to magnetic fields. Examples of shape memory alloys that respond to changes in temperature include titanium-nickel, copper-zinc-aluminum, copper-aluminum-nickel, iron-manganese-silicon, iron-nickel-aluminum, gold-cadmium, combinations of the foregoing, and the like. In certain embodiments, the shape memory alloy comprises a biocompatible material such as a titanium-nickel alloy.

Shape memory alloys exist in two distinct solid phases called martensite and austenite. The martensite phase is relatively soft and easily deformed, whereas the austenite phase is relatively stronger and less easily deformed. For example, shape memory alloys enter the austenite phase at a relatively high temperature and the martensite phase at a relatively low temperature. Shape memory alloys begin transforming to the martensite phase at a start temperature (Mₛ) and finish transforming to the martensite phase at a finish temperature (M_{f}). Similarly, such shape memory alloys begin transforming to the austenite phase at a start temperature (Aₛ) and finish transforming to the austenite phase at a finish temperature (A_{f}). Both transformations have a hysteresis. Thus, the Mₛ temperature and the A_{f} temperature are not coincident with each other, and the M_{f} temperature and the Aₛ temperature are not coincident with each other.

In certain embodiments, the shape memory alloy is processed to form a memorized shape in the austenite phase in the form of a ring or partial ring. The shape memory alloy is then cooled below the M_{f} temperature to enter the martensite phase and deformed into a larger or smaller ring. In certain such embodiments, the shape memory alloy is sufficiently malleable in the martensite phase to allow a user such as a physician to adjust the circumference of the ring in the martensite phase by hand to achieve a desired fit for a particular stomach. After the ring is attached to the stomach, the circumference of the ring can be adjusted non-invasively by heating the shape memory alloy to an activation temperature (e.g., temperatures ranging from the Aₛ temperature to the A_{f} temperature).

Thereafter, when the shape memory alloy is exposed to a temperature elevation and transformed to the austenite phase, the alloy changes in shape from the deformed shape to the memorized shape. Activation temperatures at which the shape memory alloy causes the shape of the implant to change shape can be selected and built into the implant such that collateral damage is reduced or eliminated in tissue adjacent the implant during the activation process. Examples of A_{f} temperatures for suitable shape memory alloys range between approximately 45 degrees Celsius and approximately 70 degrees Celsius. Furthermore, examples of Mₛ temperatures range between approximately 10 degrees Celsius and approximately 20 degrees Celsius, and examples of M_{f} temperatures range between approximately −1 degrees Celsius and approximately 15 degrees Celsius. The size of the implant can be changed all at once or incrementally in small steps at different times in order to achieve the adjustment necessary to produce the desired clinical result.

Certain shape memory alloys may further include a rhombohedral phase, having a rhombohedral start temperature (Rₛ) and a rhombohedral finish temperature (R_{f}), that exists between the austenite and martensite phases. An example of such a shape memory alloy is a NiTi alloy, which is commercially available from Memry Corporation (Bethel, Conn.). In certain embodiments, an example of an Rₛ temperature range is between approximately 30 degrees Celsius and approximately 50 degrees Celsius, and an example of an R_{f} temperature range is between approximately 20 degrees Celsius and approximately 35 degrees Celsius. One benefit of using a shape memory material having a rhombohedral phase is that in the rhomobohedral phase the shape memory material may experience a partial physical distortion, as compared to the generally rigid structure of the austenite phase and the generally deformable structure of the martensite phase.

Certain shape memory alloys exhibit a ferromagnetic shape memory effect wherein the shape memory alloy transforms from the martensite phase to the austenite phase when exposed to an external magnetic field. The term “ferromagnetic” as used herein is a broad term and is used in its ordinary sense and includes, without limitation, any material that easily magnetizes, such as a material having atoms that orient their electron spins to conform to an external magnetic field. Ferromagnetic materials include permanent magnets, which can be magnetized through a variety of modes, and materials, such as metals, that are attracted to permanent magnets. Ferromagnetic materials also include electromagnetic materials that are capable of being activated by an electromagnetic transmitter, such as one located outside the stomach. Furthermore, ferromagnetic materials may include one or more polymer-bonded magnets, wherein magnetic particles are bound within a polymer matrix, such as a biocompatible polymer. The magnetic materials can comprise isotropic and/or anisotropic materials, such as for example NdFeB (neodymium-iron-boron), SmCo (samarium-cobalt), ferrite and/or AlNiCo (aluminum-nickel-cobalt) particles.

Thus, an implant comprising a ferromagnetic shape memory alloy can be implanted in a first configuration having a first shape and later changed to a second configuration having a second (e.g., memorized) shape without heating the shape memory material above the Aₛ, temperature. Advantageously, nearby healthy tissue is not exposed to high temperatures that could damage the tissue. Further, since the ferromagnetic shape memory alloy does not need to be heated, the size of the implant can be adjusted more quickly and more uniformly than by heat activation.

Examples of ferromagnetic shape memory alloys include Fe—C, Fe—Pd, Fe—Mn—Si, Co—Mn, Fe—Co—Ni—Ti, Ni—Mn—Ga, Ni₂MnGa, Co—Ni—Al, and the like. Certain of these shape memory materials may also change shape in response to changes in temperature. Thus, the shape of such materials can be adjusted by exposure to a magnetic field, by changing the temperature of the material, or both.

In certain embodiments, combinations of different shape memory materials are used. For example, implants according to certain embodiments comprise a combination of shape memory polymer and shape memory alloy (e.g., NiTi). In certain such embodiments, an implant comprises a shape memory polymer tube and a shape memory alloy (e.g., NiTi) disposed within the tube. Such embodiments are flexible and allow the size and shape of the implant to be further reduced without impacting fatigue properties. In addition, or in other embodiments, shape memory polymers are used with shape memory alloys to create a bi-directional (e.g., capable of expanding and contracting) implant. Bi-directional implants can be created with a wide variety of shape memory material combinations having different characteristics.

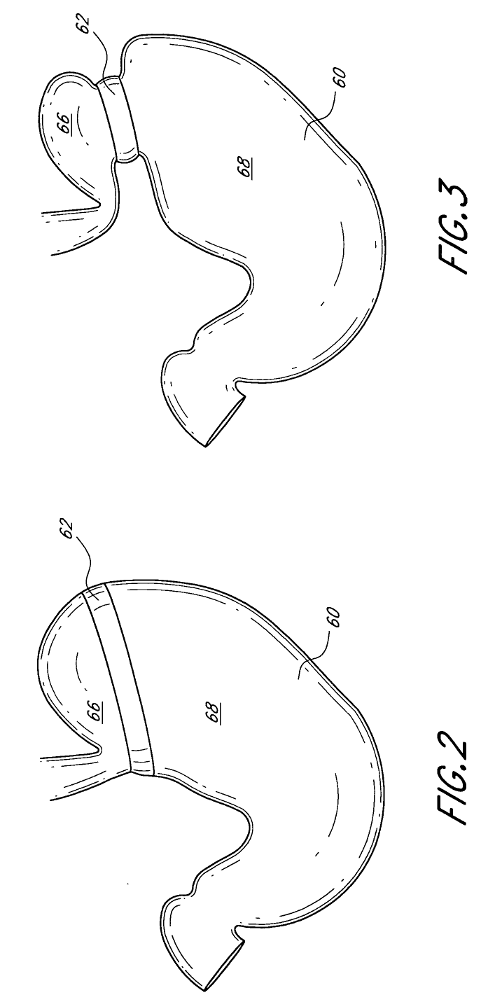

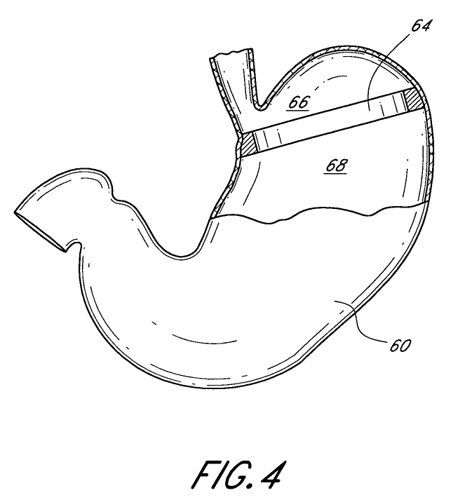

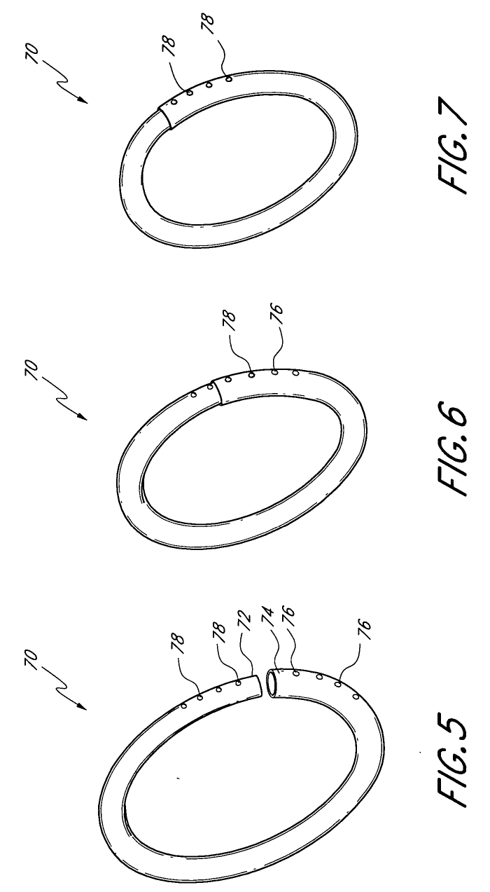

The present embodiments provide a system, method, and various devices to dynamically remodel and resize the stomach as the patient's needs change. For example, FIGS. 2 and 3 illustrate the pre- and post-adjustment configurations of a stomach 60 and one embodiment of a generally ring-shaped implant 62. In FIGS. 2 and 3 the implant 62 is configured to be disposed around the exterior surfaces of the stomach 60. FIG. 4 illustrates the pre-adjustment configuration of a stomach 60 and another embodiment of a generally ring-shaped implant 64 that is configured to be disposed within the stomach 60. The size and shape of each implant 62, 64 can be selected based upon the patient's anatomy. FIGS. 5-29, discussed in detail below, illustrate some examples of possible shapes.

FIGS. 2 and 4 illustrate the implants immediately after implantation, prior to any adjustments in the size and/or shape of the implants. In the illustrated configuration each of the generally ring-shaped implants forms a dividing line that separates the stomach into two regions. An upper region 66 includes the fundus, at least a portion of the cardia, and a portion of the body. A lower region 68 includes a portion of the body and the pylorus. Those of ordinary skill in the art will appreciate that the implants may be positioned and oriented in any of a variety of different ways from that illustrated. The exact positioning and orientation of the implants can be determined by the implanting physician according to the patient's needs.

The position of the implant relative to the stomach can be secured in any of a variety of ways. For example, sutures, staples, tacks, pins, and/or adhesives may secure the implant to the stomach. Stapling methods may include automatic or manual stapling. Adhesives may include, for example, tissue glue, heat activated glue, UV-curable glue, and room temperature or moisture activated glue. Securing and/or suturing of the various implant embodiments to the tissue can include a variety of energy sources, such as RF heating, laser, microwave, ultrasound, etc. Securing and/or suturing of the various implant embodiments to the tissue can be done all around the implant perimeter or at one or more points or segments. In certain embodiments, the implant may include one or more holes or suture rings through which sutures may pass, as described in more detail below.

FIG. 3 illustrates the stomach 60 and the external implant 62 of FIG. 2 after adjustments have been made to the size of the implant. As in FIG. 2, the generally ring-shaped implant separates the stomach into an upper region 66 and a lower region 68. The upper region forms a gastric pouch that can only hold a small amount of food. A stoma (not shown) connects the upper and lower regions. As the size of the implant decreases from the configuration of FIG. 2 to that of FIG. 3, the size of the stoma shrinks, thus limiting the rate at which food can pass from the upper stomach pouch to the lower region. Depending upon the patient's needs, the physician can activate the implant to achieve a smaller size, and thus a smaller stoma, from that illustrated in FIG. 3. Alternatively, during the activation procedure(s) the physician can stop short of the size illustrated in FIG. 3 so that the implant is configured to have a larger size, and thus a larger stoma, from that illustrated. As those of skill in the art will appreciate, the stomach and the internal implant 64 of FIG. 4 can be manipulated in a fashion similar to that just described for the external implant of FIGS. 2 and 3.

In certain embodiments the shape memory material of the implant may be bi-directional, so that it is capable of expanding and contracting. With such an embodiment, the physician can dynamically adjust the size and/or shape of the implant as the patient's needs change. For example, a patient may have a need to lose a large amount of weight quickly. In such a case it may be advantageous to shrink the implant down to a relatively small size soon after implantation. The relatively small implant would then create a relatively small stoma so that the speed at which the patient could digest food would be greatly diminished, and the patient would lose weight relatively quickly. As the patient loses weight, his or her needs may change, and the physician may need to expand the implant to create a larger stoma, and thereby increase the speed at which the patient can digest food. With a bi-directional implant, the physician could easily expand the implant using one or more of the non-invasive techniques described above.

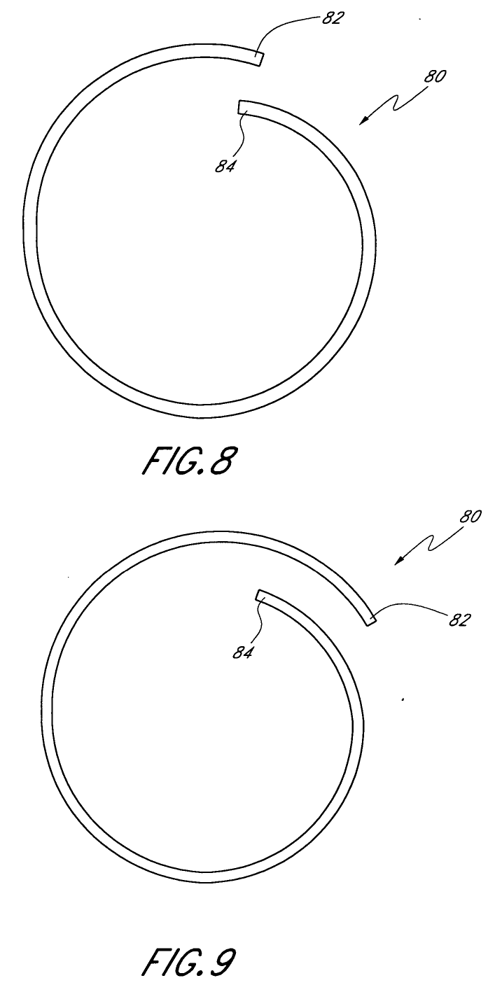

FIGS. 5-7 illustrate one embodiment of a generally ring-shaped implant 70 that may be used in the methods described above and illustrated in FIGS. 2-4. The implant 70 comprises a ring with a male end 72 that telescopically engages a female end 74. FIGS. 5-7 represent a possible time-lapse transformation of the implant 70 from a deformed shape (FIG. 5) to a memorized shape (FIG. 7). As an activating energy (such as heat, or a magnetic field, or any of the other energies described above) is applied to the implant of FIG. 5, the circumference of the implant becomes progressively smaller as the implant returns to its memorized shape, shown in FIG. 7. As the implant becomes progressively smaller, it cinches the portion of the stomach around which it is wrapped, decreasing the size of the stoma that connects the upper gastric pouch to the lower stomach region. In order to achieve a desired circumference for the implant after it has been implanted, and thus achieve a desired circumference for the stoma, the physician may halt the application of activation energy before the implant returns to its memorized shape. For example, the application of activation energy may be halted when the implant occupies the intermediate configuration of FIG. 6.

In the illustrated embodiment, the implant 70 includes retaining features that help the implant to maintain its shape after the application of activation energy has ceased. The female end 74 includes a plurality of evenly spaced holes 76. The male end 72 includes at least one protrusion 78. As activation energy is applied to the implant 70, and it contracts from the configuration of FIG. 5 toward the configuration of FIG. 7, the at least one protrusion 78 advances from one hole 76 to the next along the female end 74 as the male end 72 advances into the female end. Engagement of the at least one protrusion with each hole resists any tendency of the male end to withdraw from the female end. These retaining features thus help the implant 70 to remain in its contracted state even as the contracted stomach and/or esophagus apply pressure against the implant that might otherwise cause the implant to expand toward the configuration of FIG. 5. If the implant includes a plurality of protrusions 78 and holes 76, as illustrated, then an increasing number of protrusions and holes will engage one another as the male end advances into the female end. As the number of engaged features increases, so does the retaining power of the implant.

Those of ordinary skill in the art will appreciate that the implant 70 shown in FIGS. 5-7 is representative of a family of implants having a generally ring-shaped configuration. A variety of implants having a generally ring-shaped configuration could be produced to meet the needs of a wide variety of patients. For example, a generally ring-shaped implant may include ends that do not telescope or even overlap. FIGS. 8 and 9 illustrate another embodiment of a generally ring-shaped implant 80. The implant 80 resembles the implant shown in FIGS. 5-7, and includes first and second ends 82, 84 that overlap, but are not in contact with one another. FIG. 8 illustrates a pre-activation configuration, while FIG. 9 illustrates a post-activation configuration. As the implant 80 transforms from the pre-activation configuration (FIG. 8) to the post-activation configuration (FIG. 9), an amount of overlap of the ends 82, 84 increases as a circumference of the implant tightens.

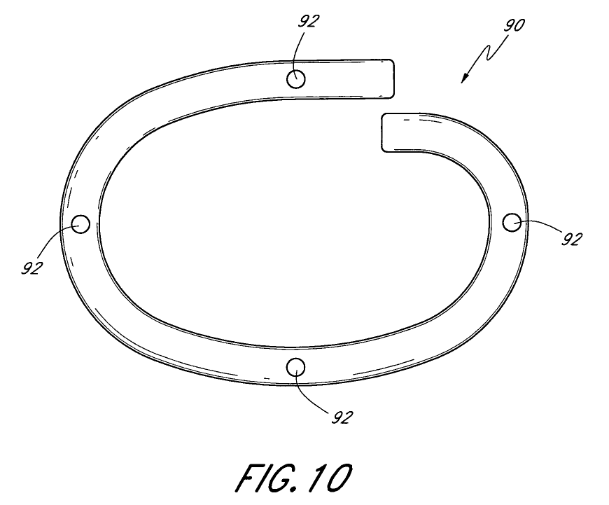

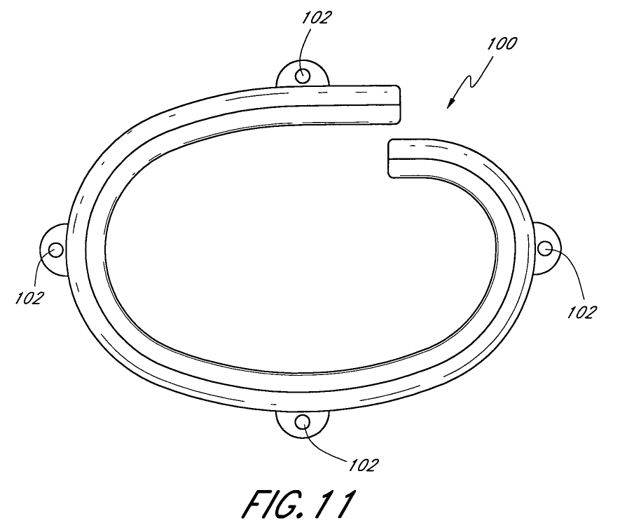

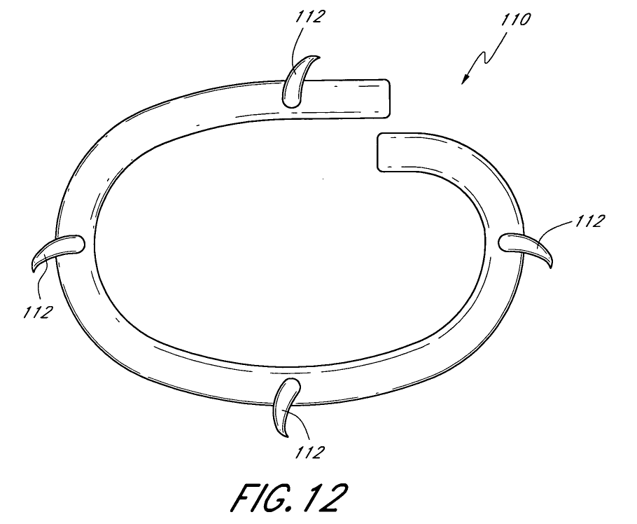

All of the embodiments of implants described herein may include features that facilitate the securement of the implant to the stomach and/or esophagus. For example, FIGS. 10-12 illustrate further embodiments of an implant 90, 100, 110 that is shaped substantially as an oval ring with overlapping ends. The implant 90 of FIG. 10 includes four evenly spaced suture holes 92, and the implant 100 of FIG. 11 includes four evenly spaced suture rings 102. In the illustrated embodiments, a longitudinal axis of each suture hole/ring extends in a direction substantially perpendicular to a plane defined by the implant. However, those of skill in the art will appreciate that the holes/rings could be oriented differently with respect to the implant. Each hole/ring may receive one or more sutures that may be used to secure the implant to the stomach. Those of ordinary skill in the art will appreciate that fewer or more suture holes/rings may be provided, and that they need not be evenly spaced. Those of ordinary skill in the art will also appreciate that suture holes/rings may be used with any of the implants described herein, and with implants of any shape or size.

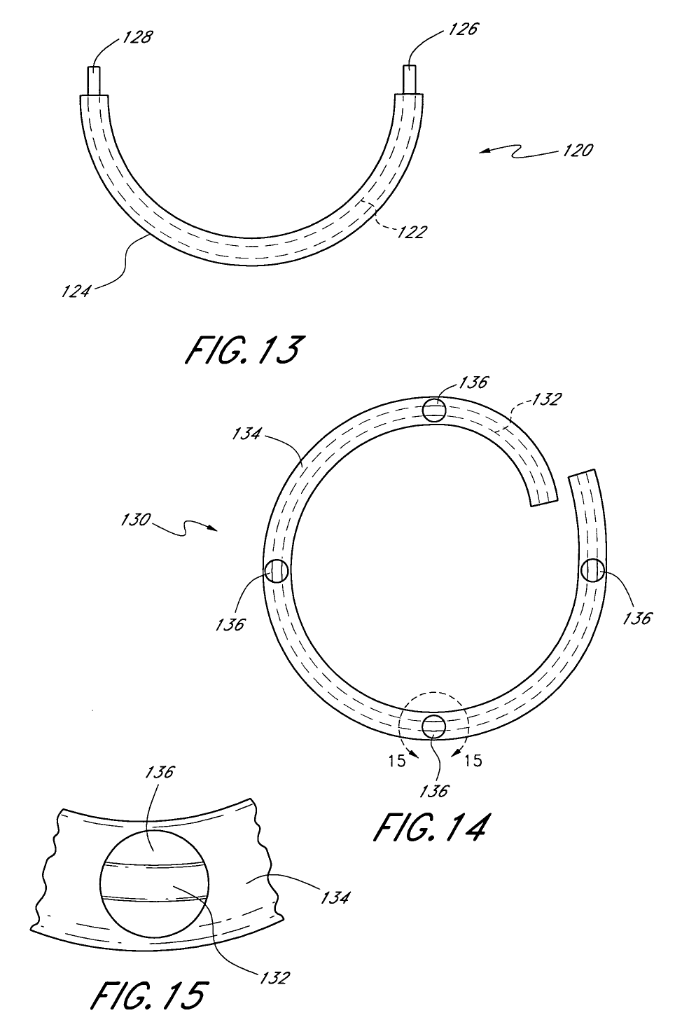

The implant 110 of FIG. 12 includes four evenly spaced hooks or barbs 112. Each hook or barb includes a sharp point that is adapted to penetrate and grip tissue. The hooks or barbs thus secure the implant 110 to the stomach. Those of ordinary skill in the art will appreciate that fewer or more hooks or barbs may be provided, and that they need not be evenly spaced. Those of ordinary skill in the art will also appreciate that hooks or barbs may be used with any of the implants described herein, and with implants of any shape or size.

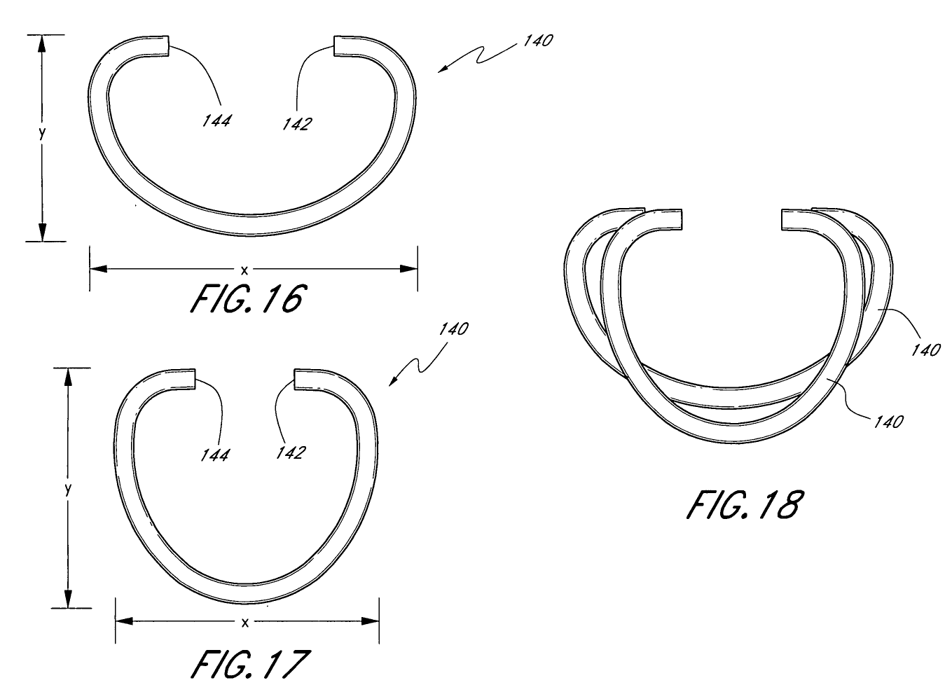

All of the embodiments of implants described herein may also include a cover. For example, FIG. 13 illustrates another embodiment of an implant 120 that is shaped substantially as a half ring, and FIG. 14 illustrates another embodiment of an implant 130 that is shaped substantially as a coiled ring with overlapping ends. Each implant 120, 130 includes a core 122, 132 formed of a shape memory material and a cover 124, 134 disposed over the core. The cover 124, 134 may be constructed of any biodegradable and/or biocompatible material, such as polytetrafluoroethylene (PTFE) and expanded polytetrafluoroethylene (ePTFE). The cover may include multiple layers, such as an insulating layer and a polymer jacket. The cover may serve as a protective barrier between the core and any surrounding tissue, and may help the implant to become integrated into the surrounding tissue. For example, the cover 124, 134 may be constructed of a porous material or a fabric. Such porous materials or fabrics can be impregnated with a time-release substance, such as anti-inflammatory drugs, anti-obesity drugs, a combination thereof, or other drugs. The cover may also comprise a lubricious coating, such as polylactic acid (PLA), that eases placement and/or removal of the implant. The cover may also aid in suturing the implant to the tissue by acting as a medium that sutures can penetrate. A surgeon implanting one of the present implant embodiments may pass a suturing needle first through the cover and then through the tissue to secure the implant to the tissue.

Depending upon the composition of the cover, it may insulate the core so that the core is less readily able to absorb activating energy and undergo a shape change. Accordingly, in the embodiment 120 of FIG. 13 at a first end and a second end of the implant the core 122 extends beyond the cover 124 to form a first exposed core portion 126 and a second exposed core portion 128. Similarly, in the embodiment of FIG. 14, the cover 134 includes four evenly spaced openings 136 that expose short lengths of the core 132. FIG. 15 illustrates a detail view of one of the openings 136 and the core 132. The exposed portions of the core may create locations where the core is readily able to absorb activating energy, which can then be conducted along the core to the non-exposed portions. The exposed portions thus provide locations at which activation energy can be focused, which both reduces energy loss during activation and reduces the likelihood that surrounding tissue might absorb unfocused activation energy and become damaged through overheating. In addition, any tissue in contact with an insulated portion of the implant is protected from absorbing heat through conduction from the implant.

FIGS. 16 and 17 illustrate another embodiment of a generally ring-shaped implant 140. The implant resembles the letter C, and includes first and second ends 142, 144 that do not overlap one another. FIG. 16 illustrates a pre-activation configuration for the implant 140, while FIG. 17 illustrates a post-activation configuration. As with all of the implant embodiments described herein, the implant may be implanted either within the stomach and/or esophagus, or around the outside of the stomach and/or esophagus. In one embodiment of a method of implantation, the implant may be implanted in the pre-activation configuration, and then activated to induce a shape change. The activation may take the form of any of the methods described above, or any equivalent method.

In the pre-activation configuration, the implant includes a width dimension x and a height dimension y. As FIG. 18 illustrates, in the post-activation configuration the width dimension x of the implant is decreased, while the height dimension y of the implant is increased. Thus, no matter where the implant is placed on or in the stomach and/or esophagus, it reshapes and resizes the stomach and/or the esophagus to alter a path of travel of food through these areas, and/or to alter a patient's ability to absorb nutrients.

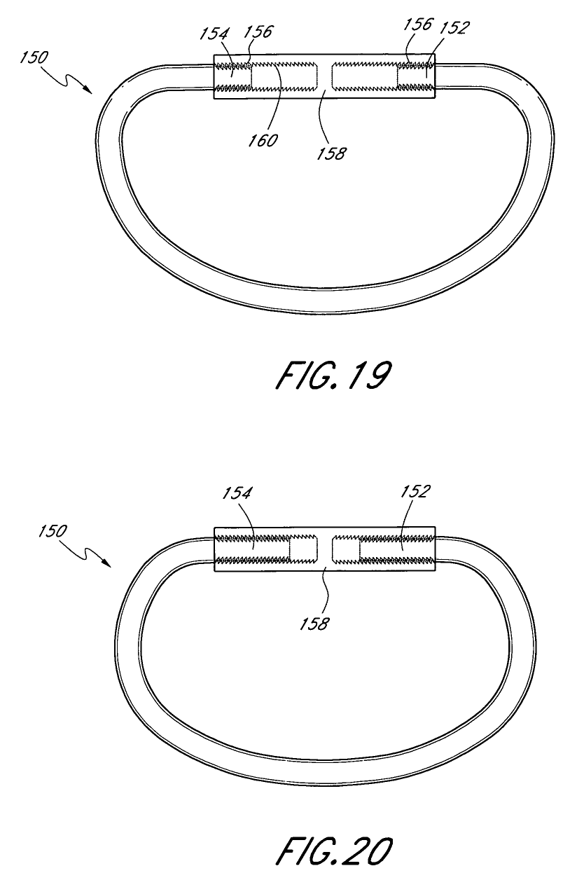

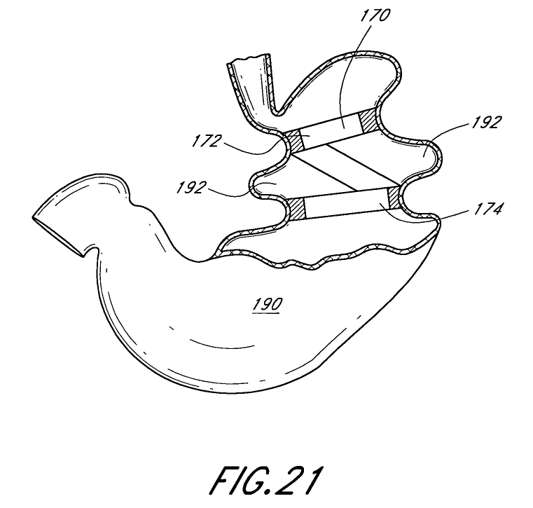

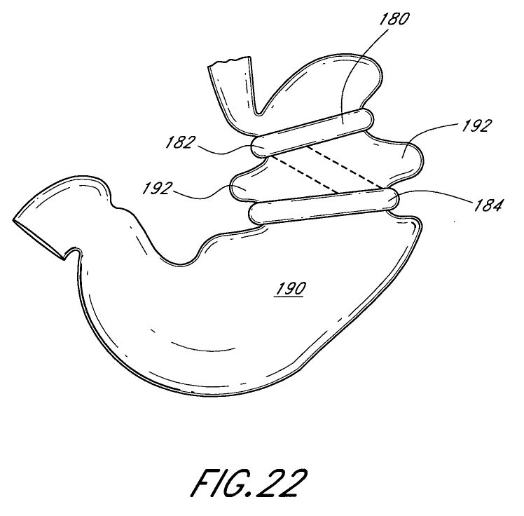

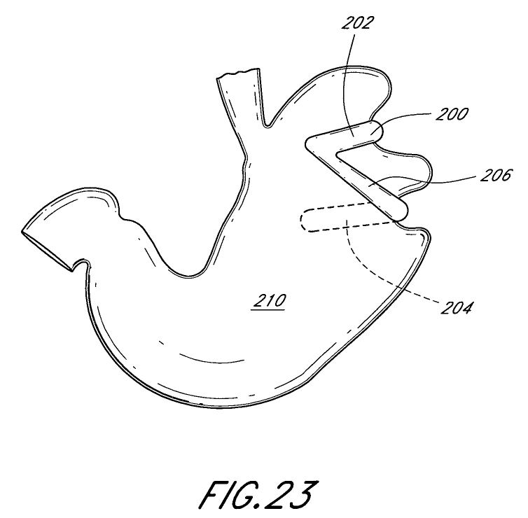

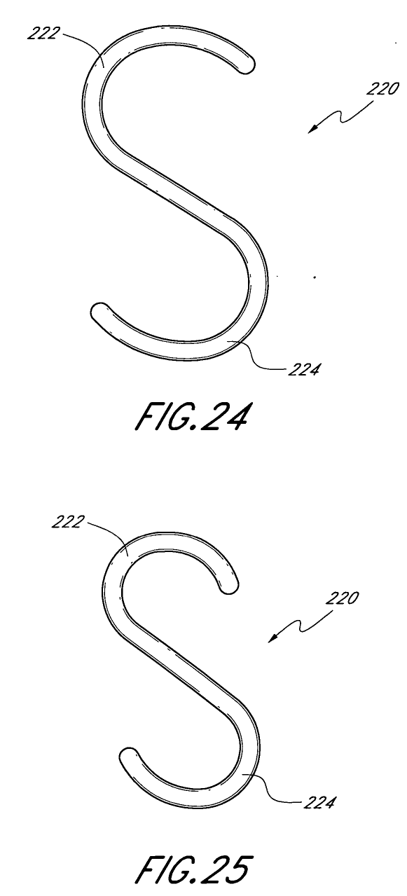

FIGS. 19 and 20 illustrate another embodiment of a generally ring-shaped implant 150. The implant 150 is similar in shape to the implant 140 shown in FIGS. 16 and 17, and includes first and second ends 152, 154 that do not overlap. FIG. 19 illustrates a pre-activation configuration, while FIG. 20 illustrates a post-activation configuration. Each of the implant ends 152, 154 includes ratchet teeth 156. A ratchet sleeve 158 receives each of the ends 152, 154. The sleeve 158 includes ratchet teeth 160 that are complementary to the teeth 156 on the implant ends. Thus, as the implant 150 progresses from the pre-activation configuration to the post-activation configuration the implant ends 152, 154 advance into the sleeve 158, and the mating ratchet teeth 156, 160 resist any tendency of the ends 152, 154 to withdraw from the sleeve 158. Because the implant ends are held firmly in the sleeve, there is less likelihood that the implant might relax and cause an unwanted change in shape of the stomach and/or esophagus.

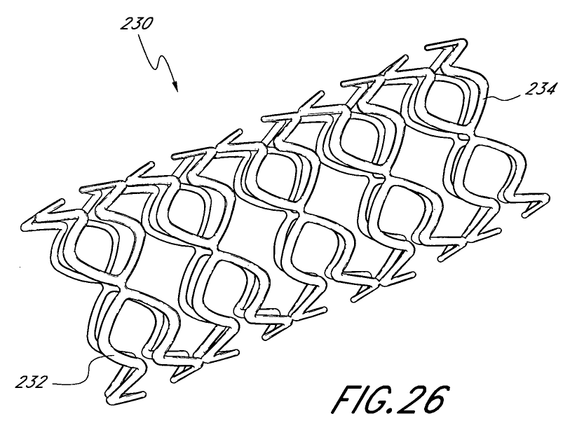

FIGS. 21 and 22 illustrate additional embodiments of the present implants 170, 180. Each of the implants 170, 180 comprises a generally helical shape with approximately two turns. As those of skill in the art will appreciate, a generally helical implant could have any number of turns.

In the illustrated embodiments, each implant 170, 180 is secured to and constricts an upper portion of the stomach 190. In FIG. 21 the implant is 170 disposed within the stomach, while in FIG. 22 the implant 180 is disposed around the outside of the stomach. As with all of the implant embodiments described herein, the implants 170, 180 of FIGS. 21 and 22 could be secured to the stomach 190 using any of the methods described herein, such as suturing, stapling, adhesives, etc., or any equivalent methods. Further, and again as with all of the implant embodiments described herein, the implants of FIGS. 21 and 22 could include apparatus to facilitate the securement of the implants, such as suture holes/rings, hooks, anchors, etc., and could include a cover.

FIGS. 21 and 22 illustrate the implants 170, 180 in a post-activation configuration. The upper turn 172, 182 and lower turn 174, 184 of each helical implant squeeze the stomach 190, constricting an upper portion of the stomach and creating a relatively narrow channel through which food can pass. The relatively narrow channel slows the passage of food, slowing the patient's digestion and making the patient feel full more quickly. The helical shape of the implants 170, 180 also shortens in length upon activation, creating bulges 192 in the stomach in the areas of the stomach that are located between adjacent turns. This deformation of the stomach creates a longer, tortuous path within the stomach for food to travel as it is being digested. The tortuous food path further reduces food intake, leading to additional weight loss benefits.

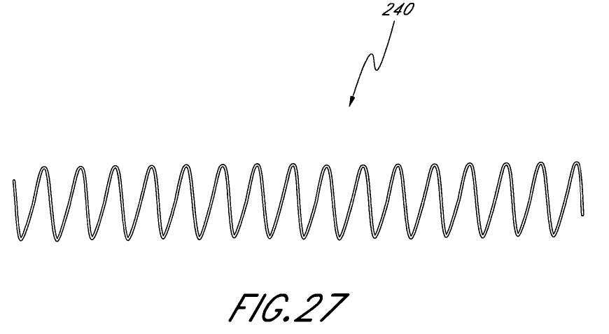

FIG. 23 illustrates another embodiment of the present implants. The implant 200 is shaped substantially as a Z, including an upper curved segment 202, a lower curved segment 204 and an intermediate segment 206 joining the upper and lower segments. The intermediate segment 204 may be substantially straight, or it may be curved. The implant 200 is adapted to be disposed on one side of the stomach 210, either on the outside as illustrated, or on the inside. In the illustrated embodiment, the implant is disposed at the upper portion of the stomach, spanning the border between the fundus and the body. Those of skill in the art will appreciate, however, that the implant could be positioned anywhere on the stomach and/or esophagus. FIG. 23 illustrates the implant 200 in a post-activation configuration. Like the helical embodiments described above, the implant is adapted to constrict the stomach/esophagus to narrow the food passageway and alter a path of travel of food through the stomach/esophagus.

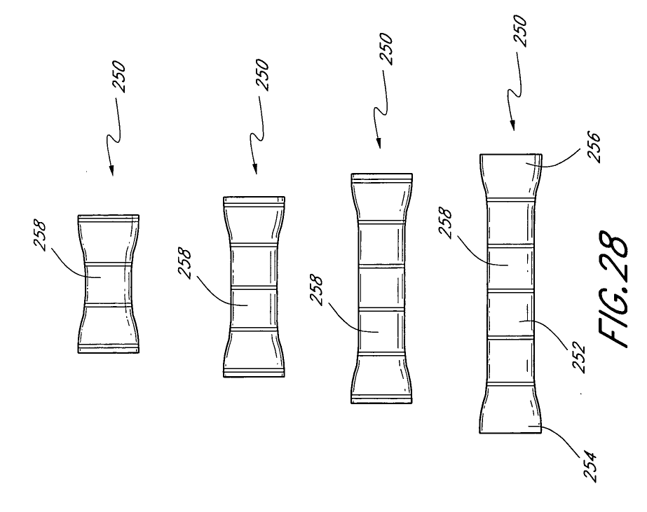

FIGS. 24 and 25 illustrate another embodiment of the present implants having a substantially S-shaped configuration. The implant 220 is adapted to be secured to one side of the stomach/esophagus, either within the stomach/esophagus or around the outside thereof. For example, the implant 220 could be positioned at the upper portion of the stomach, spanning the border between the fundus and the body. FIG. 24 illustrates the implant 220 in a pre-activation configuration, while FIG. 25 illustrates the implant in a post-activation configuration. As the implant transitions from the configuration of FIG. 24 to that of FIG. 25, an upper coil 222 and a lower coil 224 of the S tighten, thereby constricting tissue in two different places and forming upper and lower bulges in the stomach/esophagus. As with previous embodiments, the tightening of the implant constricts the stomach/esophagus to narrow the food passageway and alter a path of travel of food through the stomach/esophagus.

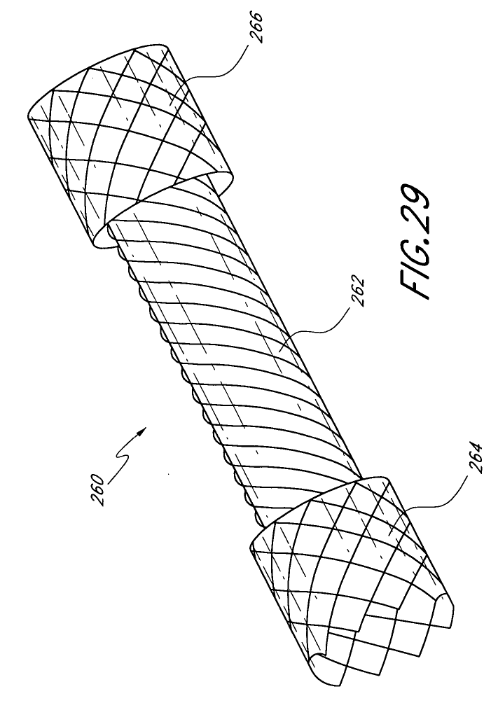

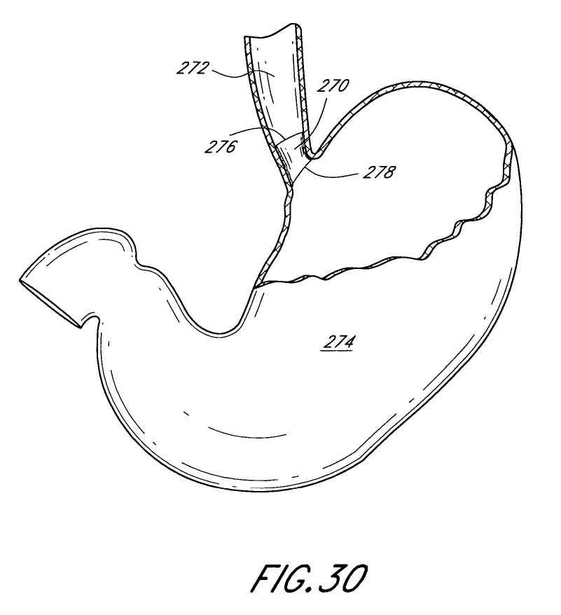

FIGS. 26-29 illustrate alternative implant configurations. These implants 230, 240, 250, 260 are modeled after typical vascular stents. For example, the implants 230, 250, 260 of FIGS. 26, 28 and 29 each resemble a tubular stent, while the implant 240 of FIG. 27 resembles a coil stent. The implants 230, 250, 260 of FIGS. 26, 28 and 29 each comprise a plurality of interconnected wire-like members that form a tubular cage structure. Those of ordinary skill in the art will appreciate that the illustrated configurations of the interconnected members are merely examples, and that implants having alternate configurations are fully equivalent to the illustrated implants.

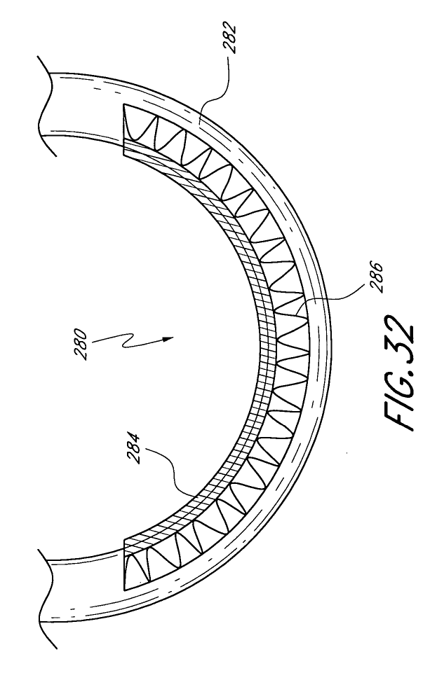

FIG. 30 illustrates, schematically, one possible configuration for implanting any of the implants of FIGS. 26-29. FIG. 30 shows a schematic configuration of an implant 270, the esophagus 272 and the stomach 274 shortly after implantation, and before any activation energy has been applied to the implant 270. In the illustrated embodiment, the implant 270 is located at the junction of the esophagus 272 and the stomach 274. An upper end 276 of the implant is located below the esophageal sphincter, while a lower end 278 of the implant extends into the stomach. Either end of the implant may be secured to the organ tissue, while portions of the implant in between the ends may also be secured to the tissue. While the illustrated implant is located within the esophagus and the stomach, those of skill in the art will appreciate that the implant could be located around the outside of these organs. Those of skill in the art will appreciate that any of the implants disclosed herein could also be located at the junction of the esophagus and the stomach. Those of skill in the art will also appreciate that the implants of FIGS. 26-29 could be implanted entirely within the stomach, or around the outside of the stomach.

When activation energy is applied to the implant 270 shown in FIG. 30, it may contract, thereby constricting the stomach/esophagus to narrow the food passageway and alter a path of travel of food through the stomach/esophagus. The extent of organ tissue constricted depends upon how much of the implant is secured to the stomach/esophagus.

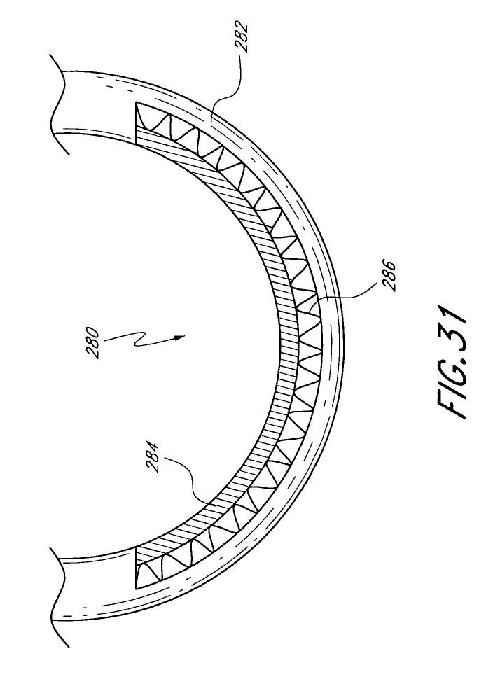

In FIG. 26, the implant 230 has a constant diameter from a first end 232 to a second end 234. In FIG. 28, the implant 250 has a constant diameter along an intermediate segment 252, then flares outwardly to a larger diameter at either end 254, 256. In FIG. 29, the implant 260 has a constant diameter along an intermediate segment 262, then abruptly transitions to a larger diameter at either end 264, 266. With the implants 250, 260 of FIGS. 28 and 29, the transition from the large opening at the proximal end 254, 264 to the relatively small intermediate section 252, 262 allows the implants to bring food slowly into the stomach, since the food will slow down at the bottleneck. Food will also exit the implant more quickly through the relatively wide distal end 256, 266.

Possible dimensions for the generally tubular implants of FIGS. 26-29 include the following. If the implant is to be positioned at the junction of the esophagus and the stomach, the implant might be between 5 mm and 50 mm in diameter, and between 20 and 200 mm in length. If the implant is to be positioned within or around the outside of the stomach, the implant might be between 20 mm and 100 mm in diameter, and between 20 and 200 mm in length.

In the embodiment 250 of FIG. 28, several different lengths of the implant are shown, and the cage-like structure of the implant is concealed by a sleeve 258. The sleeve 258 is analogous to the cover discussed above with respect to the embodiments having a shape memory core and a cover. The sleeve 258 may thus be constructed of any of the materials discussed above with respect to the cover, and share any of the same properties discussed above with respect to the cover.

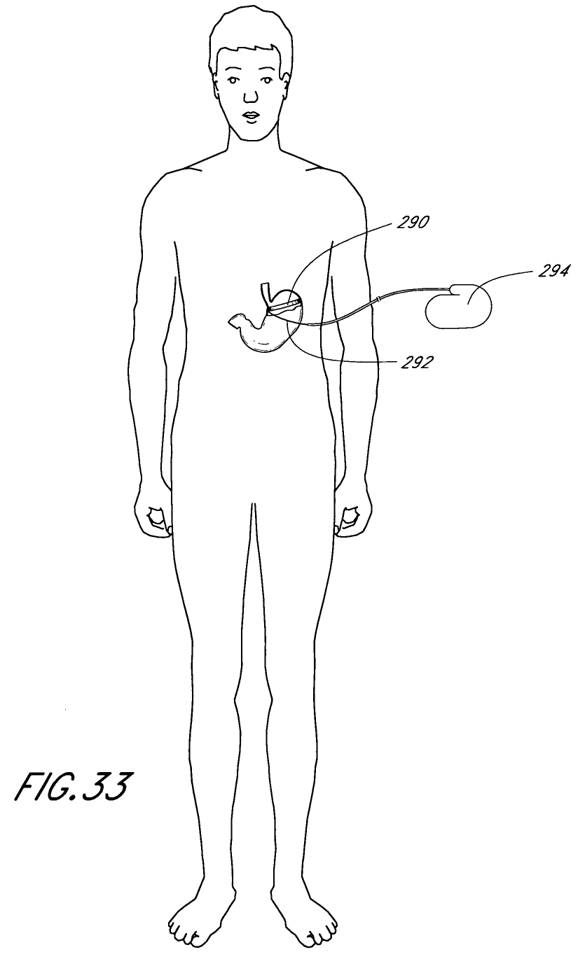

FIGS. 31 and 32 illustrate one possible configuration for any of the implants disclosed herein. The implant segment 280 includes a frame 282 constructed of a material that does not have a shape memory. For example, the frame 282 could be constructed of a metal or a polymer. Along an interior surface (a surface that will contact the stomach/esophagus) the frame 282 includes band 284 of a flexible material. For example, the band 284 could be constructed of silicone rubber. Disposed just behind the band is a layer of a shape memory material 286. In the illustrated embodiment, the shape memory material has a coiled configuration. However, those of skill in the art will appreciate that the shape memory material layer could have any configuration.

FIG. 31 illustrates the implant segment 280 in a pre-adjusted configuration, while FIG. 32 illustrates the implant segment 280 in a post-adjusted configuration. In FIG. 31 the inner band 284 is substantially flush with the inner surface of the frame 282. After the shape memory material 286 is activated, the inner band 284 is pushed outward away from the inner surface and into the configuration shown in FIG. 32. If an implant having the configuration of FIGS. 31 and 32 is disposed around the outside of a stomach/esophagus, the inner band 284 will constrict the stomach/esophagus as it is pushed away from the inner surface.

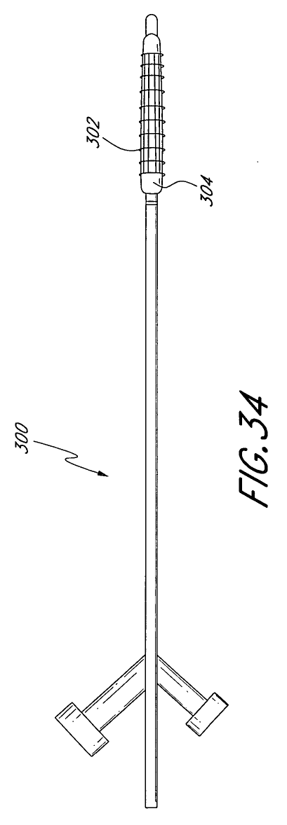

As discussed above, the size and/or configuration of any of the present implants may be adjusted post-implantation through one of many techniques, including minimally invasive techniques (endoscopic, laparoscopic, percutaneous, etc.) and completely non-invasive techniques (MRI, HIFU, inductive heating, a combination of these methods, etc.). FIG. 33 illustrates one example of a minimally invasive technique. The implant 290 may be directly connected to an electrical lead 292 that passes through the patient's skin. An external end of the lead may be connected to an electronic device 294 that is configured to generate electrical impulses. The lead 292 may transmit the impulses to the implant 290, generating activation energy within the implant in the form of heat.

Also as discussed above, the present implants may be implanted in any of a variety of ways, such as during a traditional open procedure, or endoscopically, or laparoscopically, or percutaneously, or through another type of procedure. FIG. 34 illustrates one method of implanting the present implants using a balloon catheter 300. The implant 302 may be loaded over the balloon 304, and the balloon advanced to the implantation site. Once the implant reaches the implantation site, the balloon may be inflated to expand the implant. After the balloon is deflated and removed from the implantation site, the expanded implant can be secured to the stomach/esophagus using any of the methods described above. While FIG. 34 illustrates a generally tubular implant, those of skill in the art will appreciate that the balloon catheter implantation method can be used with any of the implants described herein.

### SCOPE OF THE INVENTION

The above presents a description of the best mode contemplated for carrying out the present gastric implants and methods, and of the manner and process of making and using them, in such full, clear, concise, and exact terms as to enable any person skilled in the art to which it pertains to make and use these gastric implants and methods. These gastric implants and methods are, however, susceptible to modifications and alternate constructions from that discussed above that are fully equivalent. Consequently, these gastric implants and methods are not limited to the particular embodiments disclosed. On the contrary, these gastric implants and methods cover all modifications and alternate constructions coming within the spirit and scope of the gastric implants and methods as generally expressed by the following claims, which particularly point out and distinctly claim the subject matter of the gastric implants and methods.

What is claimed is:

1. An adjustable gastric implant configured to be implanted within a stomach and/or an esophagus or around outer surfaces of the stomach and/or the esophagus, comprising:
an implant body having at least a portion thereof formed from a material having a shape memory, the implant body being configured to transform under the influence of an activation energy from a pre-activation configuration to a post-activation configuration;
wherein the implant reshapes the stomach and/or the esophagus in transforming from the pre-activation configuration to the post-activation configuration.

2. The adjustable gastric implant of claim 1, wherein the implant is shaped substantially as at least one of a ring, an oval, a C, a D, a U, an S, a helix, a coil, a tube, a tubular cage, and a wire stent.

3. The adjustable gastric implant of claim 1, wherein the implant further comprises apparatus configured to facilitate the securement of the implant to the stomach and/or the esophagus.

4. The adjustable gastric implant of claim 3, wherein the apparatus comprises at least one of a suture hole, a suture ring, a hook, a barb, and an anchor.

5. The adjustable gastric implant of claim 1, wherein the implant body comprises:
a core having at least a portion thereof formed from a material having a shape memory; and
a cover disposed over at least a portion of the core.

6. The adjustable gastric implant of claim 1, wherein the implant further comprises apparatus configured to resist a tendency of the implant to transform from the post-activation configuration to the pre-activation configuration.

7. The adjustable gastric implant of claim 6, wherein the apparatus comprises a ratchet.

8. The adjustable gastric implant of claim 1, wherein at least the portion of the implant body that is formed from a material having a shape memory comprises at least one of a metal, a metal alloy, a nickel titanium alloy, and a shape memory polymer.

9. The adjustable gastric implant of claim 8, wherein at least the portion of the implant body that is formed from a material having a shape memory comprises at least one of Fe—C, Fe—Pd, Fe—Mn—Si, Co—Mn, Fe—Co—Ni—Ti, Ni—Mn—Ga, Ni₂MnGa, and Co—Ni—Al.

10. The adjustable gastric implant of claim 1, wherein the implant is configured to transform from the pre-activation configuration to the post-activation configuration in response to at least one of a magnetic resonance imaging energy, high-intensity focused ultrasound energy, radio frequency energy, x-ray energy, microwave energy, light energy, electric field energy, magnetic field energy, inductive heating, and conductive heating.

11. The adjustable gastric implant of claim 1, wherein the implant body comprises a frame portion and a band portion, the frame portion and the band portion each being formed of a material that does not have a shape memory.

12. The adjustable gastric implant of claim 1, wherein the implant body further comprises a shape memory portion located intermediate the frame portion and the band portion.

13. A method for treating obesity, the method comprising the steps of:
placing an adjustable gastric implant comprising a shape memory material within or around an outside surface of a patient's stomach and/or esophagus; and
applying an activation energy to the shape memory material;
wherein the activation energy induces a transformation in shape and/or size of the implant, thereby reshaping the stomach and/or esophagus.

14. The method of claim 13, wherein the implant is shaped substantially as at least one of a ring, an oval, a C, a D, a U, an S, a helix, a coil, a tube, a tubular cage, and a wire stent.

15. The method of claim 13, wherein the implant comprises at least one of a metal, a metal alloy, a nickel titanium alloy, and a shape memory polymer.

16. The method of claim 15, wherein the implant comprises at least one of Fe—C, Fe—Pd, Fe—Mn—Si, Co—Mn, Fe—Co—Ni—Ti, Ni—Mn—Ga, Ni₂MnGa, and Co—Ni—Al.

17. The method of claim 13, wherein the activation energy comprises at least one of magnetic resonance imaging energy, high-intensity focused ultrasound energy, radio frequency energy, x-ray energy, microwave energy, light energy, electric field energy, magnetic field energy, inductive heating, and conductive heating.

18. An adjustable gastric implant comprising:
means for engaging at least one of a stomach and/or an esophagus;
said means comprising a shape memory material;
said means being configured to change shape from a first configuration to a second configuration in response to an activation energy; and
said means configured to reshape said stomach and/or said esophagus in changing shape from said first configuration to said second configuration.
